(19) United States
(12) Patent Application Publication
(10) Pub. No.: US 2021/0134455 A1
(43) Pub. Date: May 6, 2021

(54) SAFE TRACKER

(71) Applicant: Christopher Smith, Foss, OK (US)

(72) Inventor: Christopher Smith, Foss, OK (US)

(21) Appl. No.: 16/675,185

(22) Filed: Nov. 5, 2019

(51) Int. Cl. G16H 40/67; G01S 19/13; A61B 5/0205; A61B 5/00

(52) U.S. Cl. CPC G16H 40/67; G01S 19/13; A61B 5/0205; A61B 5/02438; A61B 5/746; A61B 5/0015; A61B 5/681

(57) ABSTRACT

An illustrated safe tracker for determining conditions of an employee is presented. The safe tracker is useful for providing a location information, in the form of GPS data, to a computing system to track a location of an employee. The safe tracker is useful for determining medical conditions, such as heart rate, H2s levels, etc. and conveying the determined medical conditions to a computing device with an employee's name and location. The safe tracker automatically reporting the information and when an emergency condition is determined, then an alert is sent to the computing system.

### FIELD OF THE INVENTION

This invention relates to bracelets. More particularly, it relates to tracking systems installed in bracelets.

### BACKGROUND

A bracelet is an article of jewelry that is worn around the wrist. Bracelets may serve different uses, such as being worn as an ornament. When worn as ornaments, bracelets may have a supportive function to hold other items of decoration, such as charms. Medical and identity information are marked on some bracelets, such as allergy bracelets, hospital patient-identification tags, and bracelet tags for newborn babies. Bracelets may be worn to signify a certain phenomenon, such as breast cancer awareness, or for religious cultural purposes.

If a bracelet is a single, inflexible loop, it is often called a bangle. When it is worn around the ankle it is called an ankle bracelet or anklet. A boot bracelet is used to decorate boots. Colloquially, handcuffs are sometimes called bracelets. Bracelets can be manufactured from metal, leather, cloth, plastic, bead or other materials, and jewelry bracelets sometimes contain jewels, rocks, wood, shells, crystals, metal, or plastic hoops, pearls and many more materials.

A tracking system is used for the observing of persons or objects on the move and supplying a timely ordered sequence of location data for further processing. GPS has global coverage but can be hindered by line-of-sight issues caused by buildings and urban canyons. RFID is excellent and reliable indoors or in situations where close proximity to tag readers is feasible, but has limited range and still requires costly readers. RFID stands for Radio Frequency Identification. This technology uses electromagnetic waves to receive the signal from the targeting object to then save the location on a reader that can be looked at through specialized software.

Tracking systems have been very beneficial in finding lost and errand people, dogs, etc. However, in many outdoor industries, the workers may be in remote or roaming between locations. Medical conditions may occur which can cause potentially deadly circumstances for the individual. There is no bracelet currently that can monitor critical attributes of a person and when a threshold is met, the bracelet being able to alert medical staff with location of the person.

Accordingly, and in light of the foregoing, there is a need for a device that can determine critical health conditions of a person and relay the need for medical help and location of the person.

### BRIEF DESCRIPTION OF THE DRAWINGS

FIG. 1 is an illustrated view of an exemplary safe tracker.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

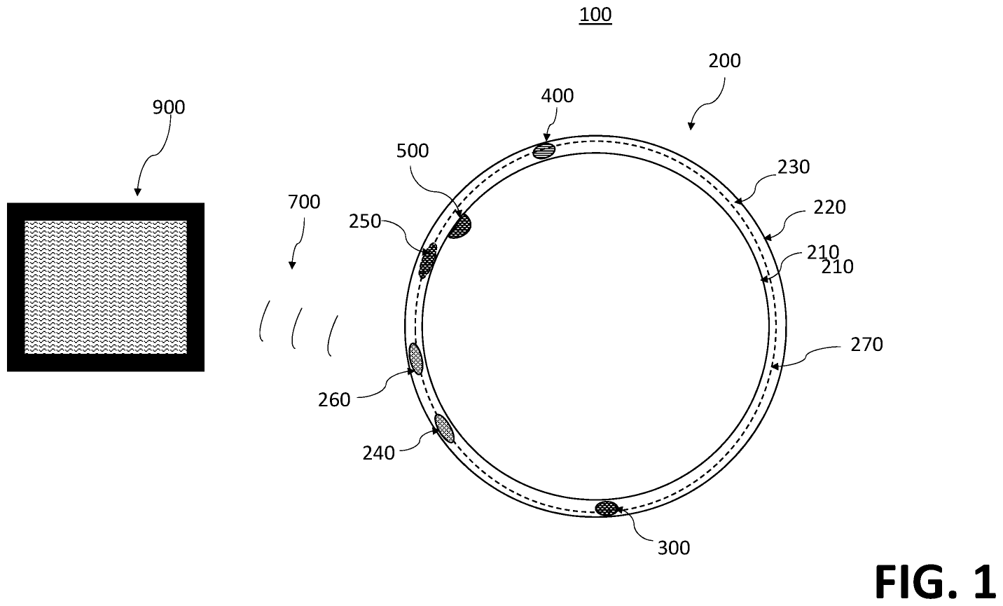

Referring to FIG. 1, an illustrated safe tracker 100 for determining conditions of an employee is presented. The safe tracker 100 is useful for providing a location information, in the form of GPS data, to a computing system to track a location of an employee. The safe tracker 100 is useful for determining medical conditions, such as heart rate, H2s levels, etc. and conveying the determined medical conditions to a computing device with an employee's name and location. The safe tracker 100 automatically reporting the information and when an emergency condition is determined, then an alert is sent to the computing system.

The safe tracker 100 has a bracelet 200, a heart rate monitor 300, a GPS module 400 and an actuator 500.

The bracelet 200 has an inside edge 210, an outside edge 220, an interior 230. The inside edge 210 is substantially near a wrist or body of a person when being worn. The outside edge 220 is visible and may have a design, pattern, etc.

The heart rate monitor 300 is coupled to a outside edge 220 of the bracelet 200. The heart rate monitor 300 receives the heart beat rate of the wearer of the safe tracker 100.

The GPS module 400 is coupled to the outside edge 220 of the bracelet 200. The GPS module 400 determines global positioning system information (GPS) such as altitude, latitude, longitude, speed, etc. The GPS module 400 is configured to be communicatively coupled to satellites.

The actuator 500 is coupled substantially near the inside edge 210 of the bracelet 200. The actuator 500 is configured to control the on/off status of the safe tracker 100. The actuator 500 is preferably a button, however other types of actuators are hereby contemplated, including, but not limited to, toggle switch, slide actuator, etc.

The interior 230 has a computing device 240, a power source 250 and a transceiver 260.

The computing device 240 is the “brains” of the safe tracker 100. The computing device 240 is communicatively coupled to the heart rate monitor 300, the GPS module 400 and the actuator 500.

The heart rate monitor 300 sends information regarding heart beat rate to the computing device 240. The computing device 240 compares the heartbeat rate to a predetermined first threshold determining whether the heartbeat rate is below the first threshold. The computing device 240 then compares the heartbeat rate to a predetermined second threshold determining whether the heartbeat rate is above the second threshold.

The GPS monitor 400 conveys location information in the for of GPS data to the computing device 210. The GPS data is then conveyed in a message to a computing system 900 via a wireless communication path 700.

The computing device 400 further calculates a H2s level and compares the H2s level to a third threshold and a fourth threshold. If the calculated H2s level is below the third threshold or above the fourth threshold, a message is sent to the computing system 900 via a wireless communication path 700.

The computing device 400 further has information stored about the employee such as employee name, employee identification number, normal heart beat rate, normal H2s level, etc. Thus, the computing device 400 has information specific to the employee. The computing device 400 sends message and information to the computing system 900 at predetermined intervals. When a heartbeat rate has been determined to be above the second threshold or below the first threshold, an emergency message is sent to the computing system 900 via the wireless communication path 700. All message sent to the computing system 900 via the wireless communication path 700 further include employee specific information such as employee name, employee identification number, location or GPS data, etc.

Further the computing device 210 may receive a command or message indicating that the employee has entered into a danger zone and requires an alert to the employee.

The power source 250 is electrically coupled to the computing device 210, the heart rate monitor 300, the GPS module 400, the actuator 500 and the transceiver 260 via a wiring apparatus 270 coupled to the interior 230 of the bracelet 200. The power source 250 is preferably a rechargeable battery, however other types of power sources are hereby incorporated, including, but not limited to, disposable battery, Nicad, etc.

The transceiver 500 is communicatively coupled to the computing device 210. The transceiver 500 is wirelessly coupled to the computing system 900 via the wireless communication path 700. The transceiver 500 is preferably compliant with an IEEE 802.11 (WiFi®) network, however other types of networks are hereby contemplated, including, but not limited to, IEEE 802.15 (Bluetooth®), 3GPP standards, 3GPP2 standards, etc.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. The safe tracker for monitoring employees, the safe tracker comprising:
a bracelet, the bracelet having an outside, an inside and an internal portion;
a power source;
a computing device, wherein the computing device being electrically coupled to the power source;
a transceiver, the transceiver being communicatively coupled to the computing device, wherein the transceiver being electrically coupled to the power source, and wherein the transceiver being wirelessly coupled to a computing system by a wireless communication path;
a GPS device, wherein the GPS device being communicative coupled to the computing device, and wherein the GPS device being electrically coupled to the power source;
a heart rate monitor, the heart rate monitor being communicative coupled to the computing device, and wherein the heart rate monitor being electrically coupled to the power source; and
an actuator, the actuator electrically coupled to the power source, and wherein the actuator coupled to the computing device.

2. The safe tracker of claim 1, wherein the computing device configured to send a command to the transceiver to send information to the computer system.

3. The safe tracker of claim 3, wherein the information being configured to have an employee identifier.

4. The safe tracker of claim 3, wherein the information being configured to have GPS information.

5. The safe tracker of claim 3, wherein the information being configured to have H2S levels.

6. The safe tracker of claim 1, wherein the computing device being configured to send information at predetermined intervals.

7. The safe tracker of claim 1, wherein when the computing device determines H2s levels being below a threshold, the computing device being configured send a command to the transceiver to send an alert message to the computer system.

8. The safe tracker of claim 1, wherein when the computing device determines heart beat levels being below a first threshold or above a second threshold, the computing device being configured send a command to the transceiver to send an alert message to the computer system.

9. The safe tracker of claim 1, wherein the computing device receiving an command to alert.

10. The safe tracker of claim 1, wherein the wireless communication path being compliant with IEEE 802.11 (Wifi®) network.
